(19) United States
(12) Patent Application Publication
(10) Pub. No.: US 2021/0321875 A1
(43) Pub. Date: Oct. 21, 2021

(54) Real-time Monitoring and Identification System Based on Bracelet Temperature Measurement and Positioning

(71) Applicant: Tsinghua University, Beijing (CN)

(72) Inventors: Bin Fang, Beijing (CN); Fuchun Sun, Beijing (CN); Huaping Liu, Beijing (CN)

(21) Appl. No.: 17/326,650

(22) Filed: May 21, 2021

Related U.S. Application Data
(63) Continuation of application No. PCT/CN2020/111409, filed on Aug. 26, 2020.

(30) Foreign Application Priority Data
Apr. 20, 2020 (CN) 2020103118706

(51) Int. Cl. A61B 5/01; H04W 4/80; H04W 8/00; H04W 4/029; H04Q 9/00; G08B 7/06; A61B 5/00; A61B 90/90; H02J 7/00

(52) U.S. Cl. CPC A61B 5/01; A61B 2562/0219; H04W 8/005; H04W 4/029; H04Q 9/00; G08B 7/06; A61B 5/7455; A61B 5/0008; A61B 90/90; A61B 5/7267; A61B 5/681; H02J 7/0047; H02J 7/0068; H04Q 2209/43; H04W 4/80

(57) ABSTRACT

The invention relates to a real-time monitoring and recognition system based on bracelet temperature measurement and positioning, comprising an ID card reader, multiple smart bracelets, multiple wireless node devices mounted at intervals in crowd gathering occasions for collecting sensing information of all smart bracelets nearby, and a cloud data processing unit for acquiring the temperature, position and motion state information of a wearer to intelligently identify the real-time body temperature health state, motion state, regional activity state, accompanying aggregation state and the like of individuals and groups through big data analysis in combination with the information, wherein each smart bracelet comprises an in-wrist temperature measurement sensor, a motion sensor, a Bluetooth sensor, a display lamp, a battery, a battery charging port, a vibration alarm and an embedded type chip processor which are arranged in a waterproof wristband; it is particularly suitable for individual and group monitoring in schools, communities, factories, etc.

### CROSS REFERENCE TO RELATED APPLICATION

The invention falls into the technical field of intelligent wearing, and is specifically relates to a real-time monitoring and identification system based on bracelet temperature measurement and positioning.

### BACKGROUND OF THE INVENTION

The outbreak of COVID-19 in 2020 has brought severe impact to the way of life and work of human beings. At present, the research results on this virus show that the fight against COVID-19 will be a drawn-out process, and the development of the epidemic will cause further increase in public's demand on temperature measurement. Currently, common temperature measuring guns consume time, manpower and have low accuracy after being used for a long time. Although box aisle infrared temperature measurement and infrared camera temperature measurement can be guaranteed in accuracy, they are expensive, low in efficiency, and bulky in size. These common products require manual operation, which is time-consuming and labor-intensive. At the same time, the existing bracelets are used for health monitoring through mobile APP, which is suitable only for individuals rather than group monitoring. Moreover, group monitoring through mobile phones can easily cause privacy leakage as personal information is stored in mobile phones. In addition, the behavior of shutting down and changing cards of individual mobile phones also brings difficulties to group monitoring.

Therefore, there is an urgent need to resolve these problems through the application of new technologies, and to design a more convenient, more accurate, more systematic, and more intelligent health monitoring management system which can effectively promote resumption of work and production as well as study.

### SUMMARY OF THE INVENTION

The objective of the invention is to overcome the shortcomings of the prior art and propose a real-time monitoring and recognition system based on bracelet temperature measurement and positioning, which can obtain the body temperature and location information of the individual in real time through a bracelet and a wireless positioning transmission system. Real-time monitoring and big data intelligent analysis are carried out on the cloud platform to identify the health and behavior of individuals and groups, which can be applied to schools, communities, factories, office buildings and other crowd gathering occasions.

To achieve the objective, the invention adopts the following technical scheme:

The invention discloses a real-time monitoring and recognition system based on bracelet temperature measurement and positioning, characterized by an ID card reader, multiple smart bracelets, multiple wireless node devices which are mounted at intervals in crowd gathering occasions, and a cloud data processing unit, where

each smart bracelet includes an in-wrist temperature measurement sensor, a motion sensor, a Bluetooth sensor, a display lamp, a battery, a battery charging port, a vibration alarm and an embedded type chip processor which are arranged in a waterproof wristband, the in-wrist temperature measurement sensor is used to measure the body temperature of the wearer; the motion sensor includes a three-axis accelerometer and a three-axis gyroscope, which are used to measure the motion state of the wearer in real time, including motion acceleration, velocity and angular velocity, so as to determine the wearer's motion state information of sitting, jumping, walking, running, and going upstairs and downstairs; vibration alarm is used to vibrate for reminding of abnormal situations, including insufficient battery charge, abnormal body temperature of the wearer, falling-off or not-wearing of the smart bracelet; the display lamp is used to indicate the normal working, charging, and low-power modes of the bracelet; the battery is used to supply power to all electrical devices in the smart bracelet, and is charged through the battery charging port; the embedded type chip processor is used to carry out signal acquisition and data processing on the in-wrist temperature measurement sensor and the motion sensor, control the display lamp to display different colors, and issue an instruction to the vibration alarm for vibration reminding when an abnormal state occurs; the Bluetooth sensor is used to send the temperature, the motion state and Bluetooth registered tag information of the wearer to the corresponding wireless node devices nearby;

the ID card reader inputs the electronic information of the wearer's identity to the embedded type chip processor of the smart bracelet through a wireless input mode, and performs registration and authentication on the smart bracelet, so as to realize association between the wearer's identity and the information of the smart bracelet;

each wireless node device is composed of a Bluetooth receiving module, which is used to search for multiple corresponding smart bracelets nearby, and collect the body temperature, the motion state information and Bluetooth signal strength of each wearer at the same time and calculate a distance between the smart bracelet and the wireless node device based on the Bluetooth signal strength; and each wireless node device sends the collected information and the calculated distance information between the smart bracelet and the wireless node device to the cloud data processing unit through the local area network;

the cloud data processing unit is used to determine the location of a plurality of corresponding smart bracelet wearers through the location of each wireless node device, and simultaneously calculate the wearer's activity track by combining the information of the motion sensor in each smart bracelet; the cloud data processing unit is used to monitor the wearer's body temperature health in real time through the wearer's body temperature information collected by the wireless node device, and is further used to obtain a group aggregation state and a regional activity state according to the comprehensive data analysis of location information and motion state information of all smart bracelets as well as carry out feature extraction on the historical data and measured data through the machine learning method, realizing real-time monitoring and identification on health state, motion state, regional activity state and accompanying aggregation state of each individual in crowd gathering occasions.

The invention has the following features and beneficial effects:

The invention provides a real-time monitoring and identification system based on bracelet temperature measurement and positioning, which is not affected by ambient light, shielding, etc., has strong adaptability, is light and exquisite, convenient to wear, precise in temperature measurement, convenient in supervision, intelligent and efficient, and is used for real-time monitoring and identification of individual health state in crowd gathering occasions. At the same time, the smart bracelet can be associated with the wearer's identity to register or unregister. Entering relatively closed places such as schools, communities, factories, office buildings, etc., you can wear it for registration, and then unregister after you leave. After being disinfected, the smart bracelet can be convenient for the wearer to register and use. The reusable way of the smart bracelet can also reduce the cost and increase the utilization rate.

### BRIEF DESCRIPTION OF THE DRAWINGS

FIG. 1 is a schematic diagram showing the structure of a real-time monitoring and identification system based on bracelet temperature measurement and positioning disclosed by the invention.

FIG. 2 is a schematic diagram showing the structure of a smart bracelet in the real-time monitoring and identification system disclosed by the invention.

In FIG. 2, 1 is an in-wrist temperature measurement sensor, 2 is a vibration alarm, 3 is an embedded type chip processor, 4 is a battery charging port, 5 is a display lamp, 6 is a battery, 7 is a motion sensor, 8 is a Bluetooth sensor and 9 is a waterproof wristband.

### DETAILED DESCRIPTION OF THE PREFERRED EMBODIMENTS

In order to clarify the objective, technical solution and advantages of the invention, the invention is further illustrated in detail in combination with the accompanying drawings and embodiments hereinafter. It should be understood that the specific embodiments described here are used to explain the invention only, and do not limit the protection scope of the invention.

In order to better understand the invention, the following describes in detail an application example of a real-time monitoring and identification system based on bracelet temperature measurement and positioning disclosed by the invention.

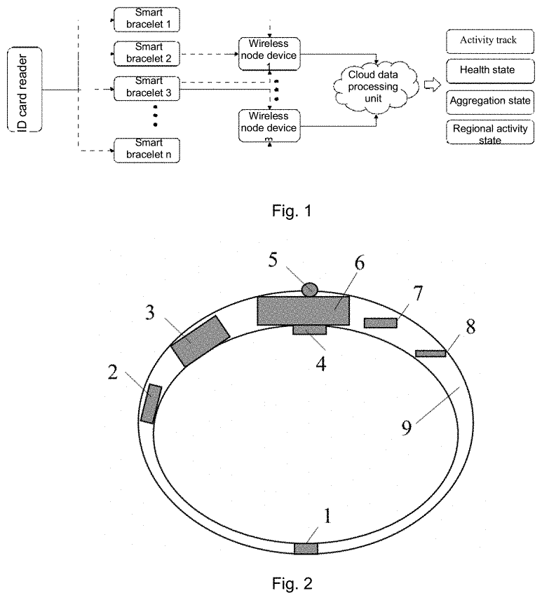

The invention discloses a real-time monitoring and identification system based on bracelet temperature measurement and positioning, with its structure as shown in FIG. 1. The real-time monitoring and identification system includes an ID card reader, multiple smart bracelets (n smart bracelets in total being arranged as shown in FIG. 1), multiple wireless node devices mounted at intervals in crowd gathering occasions (m wireless node devices in total being arranged as shown in FIG. 1) and a cloud data processing units, where:

the multiple smart bracelets are the same in structure as shown in FIG. 2, each smart bracelet includes an in-wrist temperature measurement sensor 1, a motion sensor 7, a Bluetooth sensor 8, a display lamp 5, a battery 6, a battery charging port 4, a vibration alarm 2 and an embedded type chip processor 3 which are arranged in a waterproof wristband 9, where the in-wrist temperature measurement sensor 1 is used to measure the temperature inside the wrist of the wearer, with temperature measurement accuracy being 0.1°, the motion sensor 7 includes a three-axis accelerometer and a three-axis gyroscope, which are used to measure the motion state of the wearer in real time, including motion acceleration, velocity and angular velocity, so as to determine the wearer's motion state information of sitting, jumping, walking, running, and going upstairs and downstairs; vibration alarm 2 is used to vibrate for reminding of abnormal situations, including insufficient battery charge, abnormal body temperature of the wearer, falling-off or not-wearing of the smart bracelet; the display lamp 5 is used to indicate the normal working, charging, and low-power modes of the bracelet; the battery 6 is used to supply power to all electrical devices in the smart bracelet, and is charged through the battery charging port 4; the embedded type chip processor 3 is used to carry out signal acquisition and data processing on devices such as the in-wrist temperature measurement sensor 1 and the motion sensor 7, control the display lamp to display different colors according to the state of the battery, and send an instruction to the vibration alarm 2 to carry out vibration reminding when an abnormal state occurs by analyzing information of the in-wrist temperature measurement sensor 1 and the motion sensor 7; the Bluetooth sensor 8 is used to send the temperature, the motion state and Bluetooth registered tag information of the wearer to the corresponding wireless node devices nearby, and the waterproof wristband 9 is used to protect each part and facilitate wearing the wrist

In this embodiment, the in-wrist temperature measurement sensor 1 adopts a thermometer sensor WD3703 contact type human body temperature sensor patch, the motion sensor 7 is an MPU6500 six-axis sensor, the Bluetooth sensor 8 adopts a TI CC2640R2F chip, and the embedded type chip processor 3 adopts a STM32 Embedded chip processor, where temperature and motion sensor information is processed by the embedded type chip processor, and wirelessly sent out by the Bluetooth chip, and the vibration sensor and display lamp are controlled according to the state.

The ID card reader can input the electronic information of the wearer's identity to the embedded type chip processor of the smart bracelet through the wireless input mode for registering and authenticating the smart bracelet, thereby realizing the association between the wearer's identity and the information of the smart bracelet. The ID card reader in this embodiment adopts a Huaxu HX-FDX3S ID reader.

The multiple wireless node devices are the same in structure, each wireless node device is composed of a Bluetooth receiving module, which is used to search for multiple corresponding smart bracelets nearby, and collect the body temperature, the motion state information and Bluetooth signal strength of each wearer at the same time and calculate the distance between the smart bracelet and the wireless node device based on the Bluetooth signal strength; and each wireless node device sends the collected information and the calculated distance information between the smart bracelet and the wireless node device to the cloud data processing unit through the local area network;

the cloud data processing unit processes information of each smart bracelet collected through all wireless node devices specifically as follows: the cloud data processing unit is used to determine the location of a plurality of corresponding smart bracelet wearers through the location of each wireless node device, and simultaneously calculate the wearer's activity track by combining the information of the motion sensor 7 in each smart bracelet; the cloud data processing unit is further used to monitor the wearer's body temperature health in real time through the wearer's body temperature information collected by the wireless node device, to obtain a group aggregation state and a regional activity state according to comprehensive data analysis of location information and motion state information of all smart bracelets, to carry out feature extraction on big data through the machine learning method, to carry out finer classification and identification on individual or group life or work states, such as work attention and sports health, and to predict the health state, motion state, regional activity state and accompanying aggregation state of individuals and groups in the future. In this way, safe and effective monitoring means are provided for management of many relatively closed places such as schools, communities, factories, and office buildings.

Finally, it should be noted that the parts of the invention that are not described in detail fall into the well-known technologies in the field. The above embodiments are used only to illustrate the technical solutions of the invention, not to limit them; although the invention is described in detail with reference to the foregoing embodiments, those of ordinary skill in the art should understand that they can still modify the technical solutions recorded in the foregoing embodiments, or equivalently replace some of the technical features; and such modifications or substitutions do not deviate the nature of the technical solutions from the spirit and scope of the technique embodied in the embodiments according to the invention.

What is claimed is:

1. A real-time monitoring and recognition system based on bracelet temperature measurement and positioning, comprising an ID card reader, multiple smart bracelets, multiple wireless node devices which are mounted at intervals in crowd gathering occasions, and a cloud data processing unit, wherein,
each smart bracelet comprises an in-wrist temperature measurement sensor, a motion sensor, a Bluetooth sensor, a display lamp, a battery, a battery charging port, a vibration alarm and an embedded type chip processor which are arranged in a waterproof wristband, the in-wrist temperature measurement sensor is used to measure the body temperature of the wearer; the motion sensor comprises a three-axis accelerometer and a three-axis gyroscope, which are used to measure the motion state of the wearer in real time, including motion acceleration, velocity and angular velocity, so as to determine the wearer's motion state information of sitting, jumping, walking, running, and going upstairs and downstairs; vibration alarm is used to vibrate for reminding of abnormal situations, including insufficient battery charge, abnormal body temperature of the wearer, falling-off or not-wearing of the smart bracelet; the display lamp is used to indicate the normal working, charging, and low-power modes of the bracelet; the battery is used to supply power to all electrical devices in the smart bracelet, and is charged through the battery charging port; the embedded type chip processor is used to carry out signal acquisition and data processing on the in-wrist temperature measurement sensor and the motion sensor, control the display lamp to display different colors, and send an instruction to the vibration alarm for vibration reminding when an abnormal state occurs; the Bluetooth sensor is used to send the temperature, the motion state and Bluetooth registered tag information of the wearer to the corresponding wireless node devices nearby;
the ID card reader inputs the electronic information of the wearer's identity to the embedded type chip processor of the smart bracelet through a wireless input mode, and performs registration and authentication on the smart bracelet, so as to realize association between the wearer's identity and the information of the smart bracelet;
each wireless node device is composed of a Bluetooth receiving module, which is used to search for multiple corresponding smart bracelets nearby, and collect the body temperature, the motion state information and Bluetooth signal strength of each wearer at the same time and calculate the distance between the smart bracelet and the wireless node device based on the Bluetooth signal strength; and each wireless node device sends the collected information and the calculated distance information between the smart bracelet and the wireless node device to the cloud data processing unit through the local area network;
the cloud data processing unit is used to determine the location of a plurality of corresponding smart bracelet wearers through the location of each wireless node device, and simultaneously calculate the wearer's activity track by combining the information of the motion sensor in each smart bracelet; the cloud data processing unit is used to monitor the wearer's body temperature health in real time through the wearer's body temperature information collected by the wireless node device, and is further used to obtain a group aggregation state and a regional activity state according to the comprehensive data analysis of location information and motion state information of all smart bracelets as well as carry out feature extraction on the historical data and measured data through machine learning method, realizing real-time monitoring and identification on the health state, motion state, regional activity state and accompanying aggregation state of each individual in crowd gathering occasions.
